(19) United States
(12) Patent Application Publication
(10) Pub. No.: US 2012/0018302 A1
(43) Pub. Date: Jan. 26, 2012

(54) BIOSENSOR UNIT AND BIOSENSOR SYSTEM

(75) Inventors: Yasunori SHIRAKI, Kyoto (JP); Akihiro YAMAMOTO, Kyoto (JP)

(73) Assignee: ARKRAY INC., Kyoto (JP)

(21) Appl. No.: 13/191,207

(22) Filed: Jul. 26, 2011

(30) Foreign Application Priority Data
Jul. 26, 2010 (JP) 2010-166861

(51) Int. Cl. G01N 27/327; G01N 33/50

(52) U.S. Cl. 204/403.14; 204/403.01

(57) ABSTRACT

The biosensor unit has a substrate composed of a subcutaneously retained part that is retained under the skin and a base part that is placed on the skin surface. The biosensor unit comprises a sensor part detecting numerical information regarding a substance to be measured as electric signals, a signal amplifying part amplifying the electric signals, a CPU including a calculation part A/D converting the amplified electric signals and processing them to create transmittable data, a storage storing electric signals and data, a transmission part transmitting data to an external device through optical communication, and a battery part for drive. The sensor part is provided on the subcutaneously retained part and the transmission part is provided on the base part.

### CROSS-REFERENCE TO RELATED APPLICATION

This application claims the benefit of Japanese Patent Application No. 2010-166861 filed on Jul. 26, 2010, the entire disclosure of which is incorporated by reference herein.

### FIELD

This application relates to a biosensor unit and biosensor system.

### BACKGROUND

Diabetic patients can measure their blood sugar level by SMBG (self-monitoring of blood glucose). However, the measurement requires a puncture to take a blood sample and therefore is limited to several times per day. Furthermore, it is a burden for the patients.

Recently, CGM (continuous glucose monitoring) has clinically been studied to measure the circadian blood sugar level change. In CGM, a small sensor containing electrodes and an enzyme reacting with glucose is retained under the skin to measure the glucose concentration in the blood or in the subcutaneous interstitial fluid.

The specification of U.S. Pat. No. 6,560,471 describes an analysis monitor. This analysis monitor comprises a sensor, a sensor control unit, and a display unit. The sensor is retained, for example, under the skin. A voltage is applied between an electrode in contact with glucose oxidoreductase immobilized at the tip of the sensor and another electrode, and the current value between the electrodes is measured to identify the glucose concentration. Data including that obtained by the sensor and processed by the sensor control unit are transmitted/received to/from the display unit through, for example, RF (radio frequency) wireless communication. When the sensor control unit is provided together with the display unit, data are exchanged between the sensor and sensor control unit through wireless communication.

The specification of U.S. Pat. No. 6,560,471 also recites that recent much smaller electronic parts allow the sensor itself to have a structure with a function corresponding to the sensor control unit (the control part and storage described later).

### SUMMARY

However, in the case of using radio communication as in the specification of U.S. Pat. No. 6,560,471, usable frequencies are regulated by the Radio Law. Radio frequencies assigned to medical devices are regulated also in other countries as in Japan. In other words, usable frequencies and transmission power vary depending on the country. Adjustment has to be made in each individual country. Furthermore, radio communication may influence other electronic devices such as pacemakers or be influenced by noise from other electronic devices. In order to ensure the power for communication, a specific battery level is required. In other words, if a small battery is used to make the sensor smaller, the communication may become unstable and easily influenced by noise.

The present invention is invented with view of the above circumstances and an exemplary object of the present invention is to provide a biosensor unit and biosensor system capable of stable, power-saving communication with less influence on the human body and no mutual influence with other electronic devices.

The biosensor unit according to a first exemplary object of the present invention comprises:

a sensor part retained under the skin and continuously detecting signal values regarding a target substance in the specimen;

a calculation part converting the signal values to transmittable data; and

a transmission part wirelessly transmitting the data to an external device using infrared or visible light.

Preferably, the wireless transmission performed by the transmission part utilizes a light source that is an LED, organic EL, or semiconductor laser.

Preferably, the biosensor unit comprises a reception part receiving data from an external source.

Preferably, the biosensor unit comprises a plurality of transmission parts and/or reception parts.

Preferably, the plurality of transmission parts transmit the infrared or visible light having different wavelengths;

the reception parts receive light when the plurality of transmission parts each emit infrared or visible light; and

the transmission parts transmit the data using a wavelength having the maximum light intensity and/or minimum light intensity received by the reception parts when the transmission parts transmit light.

Preferably, the biosensor unit comprises a signal amplifying part amplifying the signal values from the sensor part.

Preferably, the signal amplifying part is provided on the subcutaneously retained part that is retained under the skin.

Preferably, the biosensor unit is constructed as an integrated item having the subcutaneously retained part that is retained under the skin and a base part that is placed on the skin surface.

Preferably, the base part comprises a fixing member that is applied to the skin so as to cover the base part.

Preferably, the base part comprises a sticky adhesive layer on the surface of the base part that faces the skin.

Preferably, a biocatalyst reactive with the target substance in the specimen is placed in the sensor part and the biocatalyst produces signals corresponding to the concentration of the target substance in the specimen through reaction with the target substance.

The biosensor system according to a second exemplary aspect of the present invention comprises:

the biosensor unit according to the first exemplary aspect of the present invention; and

a data station comprising a reception part capable of receiving data transmitted from the biosensor unit.

### BRIEF DESCRIPTION OF THE DRAWINGS

A more complete understanding of this application can be obtained when the following detailed description is considered in conjunction with the following drawings, in which:

FIG. 1 is a schematic illustration showing the structure of the biosensor unit according to Embodiment 1 of the present invention;

FIG. 2 is a schematic side view of the biosensor unit shown in FIG. 1;

FIG. 3 is a schematic illustration showing the structure of the biosensor system according to Embodiment 1;

FIG. 4 is a block diagram showing the structure of the biosensor system according to Embodiment 1;

FIG. 5 is a schematic illustration showing the structure of the biosensor unit according to Embodiment 2 of the present invention;

FIG. 6 is a schematic side view of the biosensor unit shown in FIG. 5;

FIG. 7 is a schematic illustration showing the structure of the biosensor system according to Embodiment 2;

FIG. 8 is a block diagram showing the structure of the biosensor system according to Embodiment 2;

FIG. 9 is a schematic illustration showing the structure of the biosensor unit according to Modified Embodiment 1 of Embodiment 2;

FIG. 10 is a schematic illustration showing the structure of the biosensor unit according to Modified Embodiment 2 of Embodiment 2;

FIG. 11 is a schematic side view showing the structure of the biosensor unit according to Modified Embodiment 3 of Embodiment 2;

FIG. 12 is a flowchart of the biosensor system according to Modified Embodiment 3 of Embodiment 2;

FIG. 13 is a schematic illustration showing the structure of the biosensor unit according to Embodiment 3 of the present invention; and

FIG. 14 is a schematic side view of the biosensor unit shown in FIG. 13.

### DETAILED DESCRIPTION

### Embodiment 1

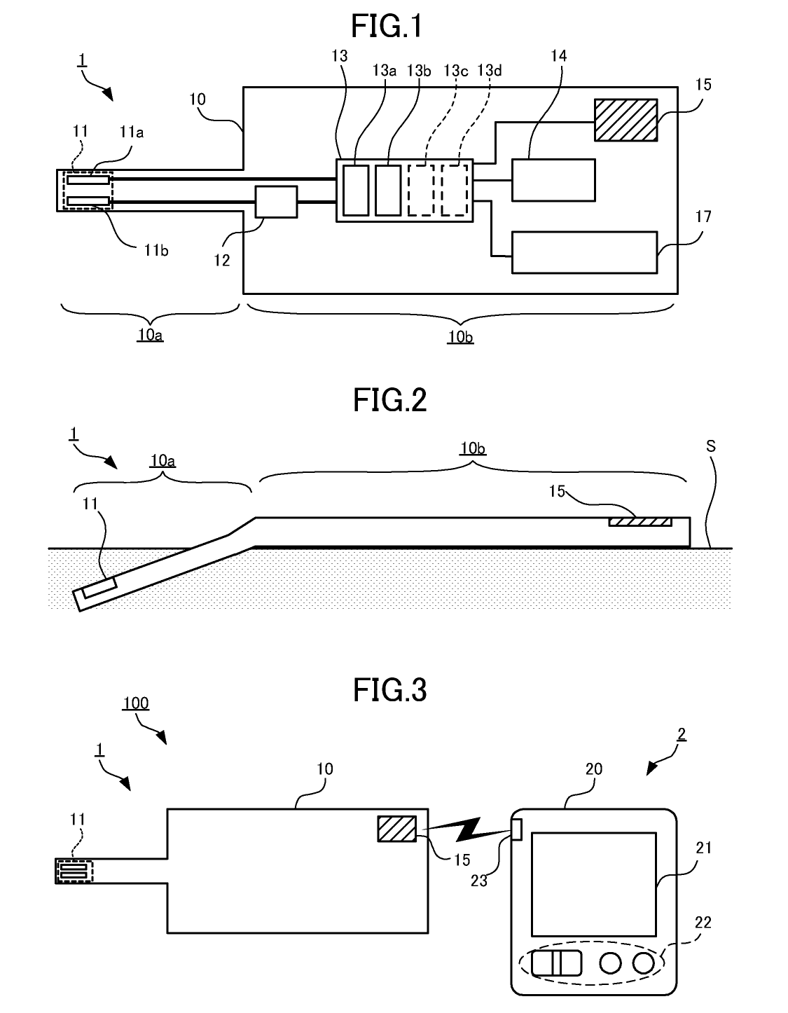

FIG. 1 is a schematic illustration showing the structure of the biosensor unit according to Embodiment 1 of the present invention. FIG. 2 is a schematic side view of the biosensor unit shown in FIG. 1. A biosensor unit 1 is a device measuring numerical information regarding a substance in the blood or interstitial fluid under the skin. Its functional parts are electrically connected by wiring.

The biosensor unit 1 has a substrate 10, a part of which can be retained under the skin S. The substrate 10 is constructed as a continuous, integrated item having a subcutaneously retained part 10a that is retained under the skin S and a base part 10b that is placed on the surface of the skin S. The biosensor unit 1 comprises a sensor part 11, a signal amplifying part 12, a CPU (central processing unit) 13, a storage 14, a transmission part 15, and a battery part 17 on the substrate 10.

For example, when the target substance of the biosensor unit 1 in the specimen is glucose contained in the blood or interstitial fluid, the biocatalyst is glucose oxidoreductase. In such a case, when a voltage is applied to the sensor part 11, the glucose oxidoreductase oxidizes glucose in the blood or interstitial fluid and electrons are retrieved. The signal value regarding the target substance in the specimen is obtained as the response current value (or the voltage value converted from the response current value) based on the quantity of electrons. The response current value is a numerical value based on the concentration of the target substance in the specimen (the glucose concentration in blood or interstitial fluid). Besides measurement of the glucose concentration, applications include measurement of lactate concentration in the test material using lactate oxidase. However, the case in which the biosensor unit 1 is a glucose sensor will be described hereafter by way of example.

The biosensor unit 1 has, for example, a total length of approximately 2 mm to 50 mm and a thickness of 1 mm or smaller. The width is not particularly restricted. However, it has a width large enough to mount the functional parts and desirably as small as possible. Desirably, the subcutaneously retained part 10a comprising the sensor part 11 is as small as possible in cross section to be inserted into the skin S. The subcutaneously retained part 10a has a width of 5 mm or smaller. The length of the subcutaneously retained part 10a is determined based on the position of the measuring target of the biosensor unit 1, namely the depth at which the subcutaneous interstitial fluid or blood is collected, and the subcutaneous insertion direction for use of the biosensor unit 1. It is preferable that the subcutaneously retained part 10a has a specific length or larger so that the sensor part 11 does not easily come off once it is subcutaneously retained. The width of the base part 10b is equal to the size (width) of the biosensor unit 1. For example, the biosensor unit 1 according to this embodiment shown in FIG. 1 has a total length of 35 mm, a width of 10 mm, and a uniform thickness of 1 mm. The subcutaneously retained part 10a has a length of 15 mm and a width of 0.5 mm. The base part 10b has a length of 20 mm and a width of 10 mm.

In the biosensor unit 1, the functional parts on the substrate 10 are electrically connected by wiring and further electrically connected to electrodes (a counter electrode 11a and a work electrode 11b) provided in the sensor part 11. For example, the electrodes and wiring can be formed on the substrate 10 by vapor deposition, sputtering, printing (screen printing or gravure printing), or transfer. The wiring and functional parts can be connected by wire bonding, metal bumps, or conductive adhesive.

An outer film (not shown) is further formed on the substrate 10. The outer film transmits a measuring target such as glucose well. Therefore, the outer film prevents biological reaction with foreign substances such as the sensor part 11 without affecting the measurement by the sensor part 11. The outer film does not need to be applied on the base part 10b as long as it covers the subcutaneously retained part 10a. The outer film preferably consists of a biocompatible material and is formed by spin coating, dip coating, or drop coating. Materials usable for the outer film include polyurethane, silicon polymers (polysiloxane), cellulose acetate, hydrogel, polyvinyl alcohol, HEMA (hydroxylethylmethacrylate), and copolymers containing them.

The substrate 10 of the biosensor unit 1 is preferably made of an insulating, flexible, and biocompatible material, including thermoplastic resins such as polyethylene phthalate (PET), polypropylene (PP), and polyethylene (PE) and thermosetting resins such as polyimide resin and epoxy resin. It is preferable that the substrate 10 is insulating because it supports the electrodes of the sensor part 11. Furthermore, it is preferable that the substrate 10 is flexible because the sensor part 11 is provided on the subcutaneously retained part 10a, the base part 10b is placed on the surface of the skin S, and the subcutaneously retained part 10a and base part 10b are constructed as an integrated item. Furthermore, it is preferable that the substrate 10 is biocompatible because the subcutaneously retained part 10a is retained in the living body for use.

The subcutaneously retained part 10a comprising the sensor part 11 of the biosensor unit 1 is retained under the skin S using an existing puncture tool. More specifically, in an existing method, the biosensor is placed in a hollow needle and the needle is inserted into the skin; then, only the needle is removed from the skin so that the sensor is retained in the skin. Similarly, it is possible to insert a hollow needle in which the biosensor unit 1 is placed into the skin S and then remove only the needle from the skin S so that the biosensor unit 1 is retained in the skin S.

The sensor part 11 is provided on the subcutaneously retained part 10a and preferably situated as close to the tip of the biosensor unit 1 as possible. The sensor part 11 comprises a biocatalyst that can be identified by the measuring target substance.

The case in which the measuring target substance is glucose and the biocatalyst is glucose oxidoreductase is described hereafter by way of example. The sensor part 11 of the biosensor unit 1 has a work electrode 11b and a counter electrode 11a for applying a voltage. A glucose oxidoreductase is immobilized on a part or the entire surface of the work electrode 11. Glucose in the blood or interstitial fluid contacts the oxidoreductase-immobilized part and the glucose is oxidized when a voltage is applied between the work electrode 11b and counter electrode 11a (electrons are retrieved). The quantity of electrons supplied to the work electrode 11b is measured as the response current value.

Examples of usable glucose oxidoreductase for measuring the glucose concentration in this embodiment include glucose oxidase (GOD) and glucose dehydrogenase (GDH). The glucose oxidoreductase can be immobilized by a known technique such as MPC polymer and protein membrane techniques. The MPC polymer can be a polymer obtained by introducing a silane coupling agent into a phospholipid polymer containing, for example, a polymerized gel, polyacrynoleamide, or phosphorus.

The signal amplifying part 12 amplifies electric signals detected by the sensor part 11. The signal amplifying part 12 allows the detection even if the electric signals detected by the sensor part 11 are weak. Furthermore, influence of external noise can be reduced. With the signal amplifying part 12 situated as close to the sensor part 11 as possible, influence of noise can further be reduced.

The CPU 13 comprises a calculation part 13a A/D converting the amplified electric signals and processing them to create transmittable data and a control part 13b controlling the functional parts of the biosensor unit 1. A clock part 13c or a reference voltage source part 13d can also be provided in addition to the calculation part 13a and control part 13b. The microcomputer or quartz oscillator of the clock part 13c can be used for timing by time difference calculation. The reference voltage source part 13d is provided to apply a voltage to the sensor part 11.

The storage 14 stores unprocessed electric signals, calculated data, and data necessary for calculation for the calculation part 13a. The storage 14 further stores conditions necessary for measurement, or conditions regarding the control of the functional parts of the biosensor unit 1. Data necessary for calculation are, for example, calibration curve information for converting a current value to a glucose concentration or calibration formulae for further calibrating the glucose concentration obtained from the calibration curve. The calculation part 13a may perform calculation after converting the response current value, which is numerical information regarding the substance, to a glucose concentration.

The transmission part 15 is a light source for optical communication. Data calculated in the calculation part 13a of the CPU 13 are transmitted to a data station such as a small device comprising a corresponding reception part. The transmission part 15 modulates and transmits light according to data to transmit. The reception part receives the modulated light and demodulates it. For example, the transmission part 15 consists of a light source that is an LED, organic EL, or semiconductor laser, and performs communication by infrared or visible light.

Using a wavelength in a range from the infrared to visible light, the transmission part 15 performs safe communication with no influence on the living body, and there is no electromagnetic wave affecting other devices. Furthermore, electric wave control or spatial resolution with a significantly high directionality is available and the communication range is visible, whereby the operation is advantageously easy.

Preferably, the transmission part 15 transmits data each time the sensor part 11 performs measurement. Furthermore, data are stored and retained in the storage 14. In addition to the transmission upon each measurement, data may collectively be transmitted after a given quantity of data are accumulated. If some failure, such as data reception failure, has occurred to the data receiving side, missing data can be prevented by storing and retaining the data.

The battery part 17 consists of a primary battery. For example, a disposable drive battery or electric double layer capacitor can be used. Using optical communication by an LED, the transmission part 15 performs power-saving communication and the battery part 17 is required to provide a smaller quantity of power. Then, the battery part 17 can be smaller and the biosensor unit 1 can have a prolonged continuous run-time.

For example, using the power from the battery part 17, the biosensor unit 1 can continuously perform the measurement at the sensor part 11 for three days to two weeks. The continuously measured glucose concentration can not only continuously be transmitted but also once be accumulated in the storage 14 and then the accumulated data can be transmitted, for example, every five to 30 minutes.

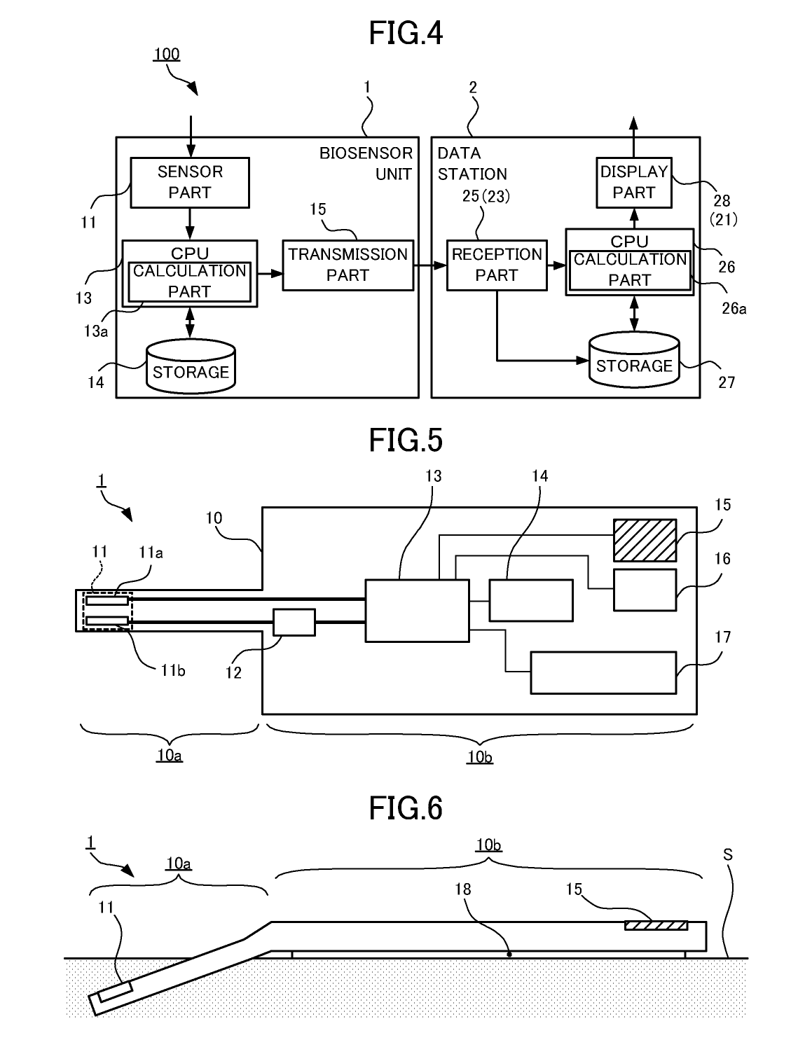

FIG. 3 is a schematic illustration showing the structure of the biosensor system according to Embodiment 1. FIG. 4 is a block diagram showing the structure of the biosensor system according to Embodiment 1. A biosensor system 100 will more specifically be described hereafter with reference to FIGS. 1 to 4. The biosensor unit 1 is used in combination with a data station 2, not alone.

The biosensor system 100 is composed of the biosensor unit 1 and a data station 2. The user of the biosensor system 100 retains the biosensor unit 1 under the skin and configures the settings so that data detected by the biosensor unit 1 are displayed on a display screen 21 of the data station 2 at given time intervals.

The data station 2 comprises a station body 20, a display screen 21, operation buttons 22, and a reception port 23. Data transmitted from the transmission part 15 of the biosensor unit 1 are received at a station reception part 25 of the data station 2 via the reception port 23 of the data station 2. The received data are processed by a station CPU 26 (including a station calculation part 26a). Then, the data are output via a display part 28 and displayed on the display screen 21 of the station body 20. The user can use the biosensor system 100 including the above series of operations of the biosensor unit 1 and data station 2.

First, the user detects data using the biosensor unit 1. The sensor part 11 of the biosensor unit 1 detects electric signals for measuring the glucose concentration in the blood or interstitial fluid. The electric signals detected by the sensor part 11 are sent to the signal amplifying part 12. The signal amplifying part 12 amplifies the electric signals and sends the amplified electric signals to the CPU 13. Then, the CPU 13 A/D converts the electric signals amplified by the signal amplifying part 12 and performs calculation to create data in a transmittable format.

The transmission part 15 of the biosensor unit 1 transmits the transmittable glucose concentration data calculated by the CPU 13 to the data station 2 comprising the corresponding station reception part 25. The data station 2 receives the data transmitted from the biosensor unit 1 at the station reception part 25 via the reception port 23.

In doing so, the transmission part 15 modulates and transmits light according to the data to transmit. The station reception part 25 receives the modulated light and demodulates it. For example, the transmission part 15 consists of a light source that is an LED, organic EL, or semiconductor laser, and performs communication by infrared or visible light. The reception port 23 is made capable of receiving the light used in the communication.

The data station 2 performs calculation on the received data at the station CPU 26 and stores them in the station storage 27. The station storage 27 can store various contents such as calculation results and conditions regarding settings of the data station 2 in addition to data. The station CPU 26 controls not only the station calculation part 26a but also the functional parts of the data station 2 and performs various necessary operations. Then, external output is made via the display part 28.

A wavelength in a range from the infrared to visible light is used for communication between the transmission part 15 of the biosensor unit 1 and the station reception part 25 of the data station 2. Therefore, the communication is performed safely with no influence on the living body and there is no electromagnetic wave affecting other devices. Furthermore, electric wave control or spatial resolution with a significantly high directionality is available and the communication range is visible, whereby the operation is advantageously easy.

As described above, the biosensor unit and biosensor system according to Embodiment 1 provide stable, power-saving communication having less influence on the human body and no mutual influence with other devices.

In Japan, radio frequencies are regulated by the Radio Law. In other counties, usable radio frequencies vary depending on the country and the communication is restricted, causing failures. However, use of optical data communication eliminates the necessity of consideration of the communication frequency and any setting is available.

Furthermore, unlike electric waves, influence of noise can be reduced and highly accurate measurement is available. Furthermore, there is no influence on and from other devices, whereby there is no risk of noise or erroneous operation; the biosensor unit and biosensor system can be used without worries.

Furthermore, the risk of influence on the human body is reduced and there is no influence of electric waves on other devices. Therefore, for example, even a person with cardiac pacemaker can use the biosensor system without worries. Furthermore, use of an LED for optical communication leads to power-saving communication. Consequently, the battery can be reduced in size and weight or provide prolonged continuous use. Then, the biosensor unit can be reduced in size and weight and/or provide prolonged continuous use.

### Embodiment 2

FIG. 5 is a schematic illustration showing the structure of the biosensor unit according to Embodiment 2 of the present invention. FIG. 6 is a schematic side view of the biosensor unit shown in FIG. 5. This biosensor unit has the same basic structure as the biosensor unit 1 according to Embodiment 1. However, a reception part 16 is additionally provided.

The reception part 16 is a reception device such as a photodiode (PD) or phototransistor (PTr) and capable of receiving data from an external source. The reception part 16 is a small device having a reception function corresponding to the transmission part 15 and receives data transmitted from a data station comprising a light source for optical communication. The reception part 16 can receive infrared or visible light produced by a light source that is an LED, organic EL, or semiconductor laser. Provision of the reception part 16 in addition to the transmission part 15 makes it possible to transmit/receive data to/from an external device.

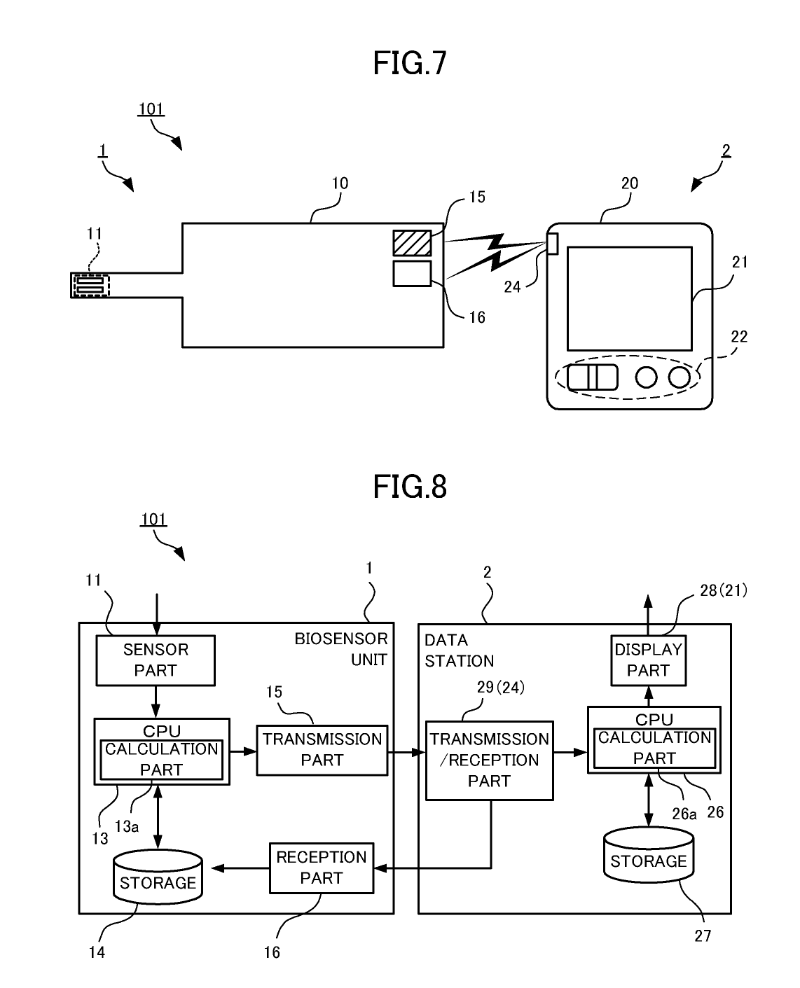

FIG. 7 is a schematic illustration showing the structure of the biosensor system according to Embodiment 2. FIG. 8 is a block diagram showing the structure of the biosensor system according to Embodiment 2. A biosensor system 101 has the same basic structure as the biosensor unit 1 and biosensor system 100 according to Embodiment 1.

The biosensor unit 1 comprises the reception part 16 in addition to the structure of the biosensor unit 1 according to Embodiment 1 described with reference to FIGS. 5 and 6. The biosensor system 101 has the same basic structure as the biosensor system 100 according to Embodiment 1. However, the biosensor unit 1 is additionally provided with the reception part 16 and the data station 2 is provided with a station transmission/reception part 29 (transmission/reception port 24) in place of the station reception part 25 (reception port 23).

The station transmission/reception part 29 of the data station 2 can receive data transmitted from the transmission part 15 of the biosensor unit 1 and transmit data to the reception part 16 of the biosensor unit 1. The biosensor unit 1 and data station 2 transmit/receive data through optical communication. For transmission, a light source that is an LED, organic EL, or semiconductor is provided for communication by infrared or visible light. For reception, a reception device such as a photodiode is provided.

Data can be transmitted/received between the biosensor unit 1 and data station 2, whereby measurement conditions or settings in the biosensor unit 1 can be changed on an arbitrary basis. Change in the measurement conditions or settings include rewriting of calculation formulae necessary for calculation by the biosensor unit 1, change of the measurement frequency, and calling for stored and retained data. Compared with the biosensor unit 1 according to Embodiment 1 having only the data transmission function, the biosensor unit 1 having the data transmission/reception function allows for maintenance of the biosensor system 101. Including adjustment of the measurement frequency, the degree of freedom in using the biosensor system 101 is improved and the user can use the biosensor system 101 tailored to meet his/her requests. Furthermore, the calculation formulae can be rewritten, whereby more accurate conversion to the glucose concentration can be done.

The biosensor unit 1 according to Embodiment 2 comprises an adhesive layer 18 on the surface of the base part 10b that faces the skin S. The adhesive layer 18 secures the base part 10b on the skin S, namely secures the biosensor unit 1 on the skin S. Then, the risk of the biosensor unit 1 coming off is reduced. Furthermore, the space between the skin S and biosensor unit 1 is eliminated, reducing the risk of the area where the biosensor unit 1 is applied being caught by an external object. The adhesive layer 18 desirably has low impact on the skin S because it makes direct contact with the skin S in a large area. For example, a hydrogel adhesive or silicon adhesive is used.

### Modified Embodiments of Embodiment 2

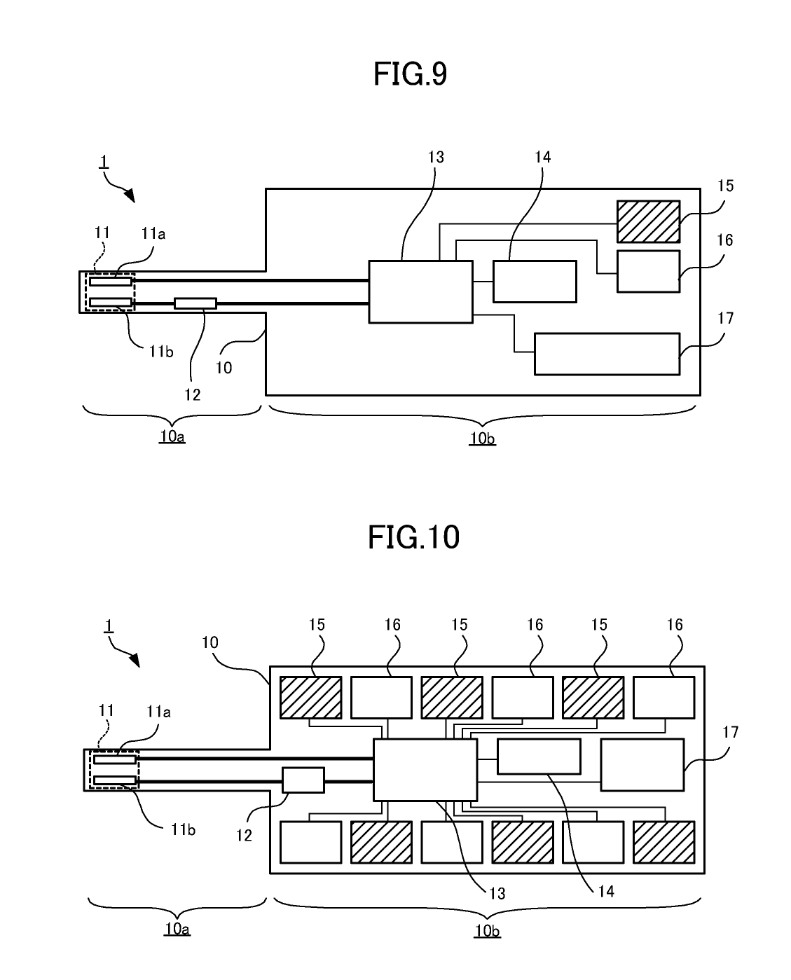

FIG. 9 is a schematic illustration showing the structure of the biosensor unit according to Modified Embodiment 1 of Embodiment 2. FIG. 10 is a schematic illustration showing the structure of the biosensor unit according to Modified Embodiment 2 of Embodiment 2.

The biosensor unit 1 shown in FIG. 9 comprises the signal amplifying part 12 on the subcutaneously retained part 10a, not on the base part 10b. Provided on the subcutaneously retained part 10a, the signal amplifying part 12 is situated closer to the sensor part 11 and amplifies electric signals detected by the sensor part 11 under much less influence of noise.

The biosensor unit 1 shown in FIG. 10 comprises multiple transmission parts 15 and reception parts 16. The multiple parts provide high directionality and further ensure data transmission/reception. The figure shows multiple transmission parts 15 and reception parts 16. However, there may be no reception part 16, or multiple transmission parts 15 and one reception part 16 may be provided and vice versa.

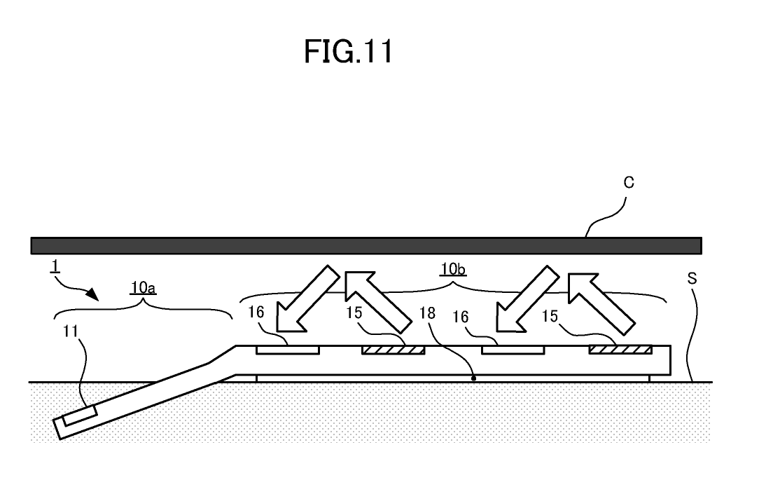

FIG. 11 is a schematic side view showing the structure of the biosensor unit according to Modified Embodiment 3 of Embodiment 2. The biosensor unit 1 shown in FIG. 11 comprises multiple transmission parts 15 and reception parts 16 as in Modified Embodiment 2 of Embodiment 2. Generally, the biosensor unit 1 is used with clothes C on. Therefore, the transmission parts 15 according to Modified Embodiment 3 of Embodiment 2 use light emitting elements of different wavelengths for transmission and the reception parts 16 are designed to receive the corresponding wavelengths.

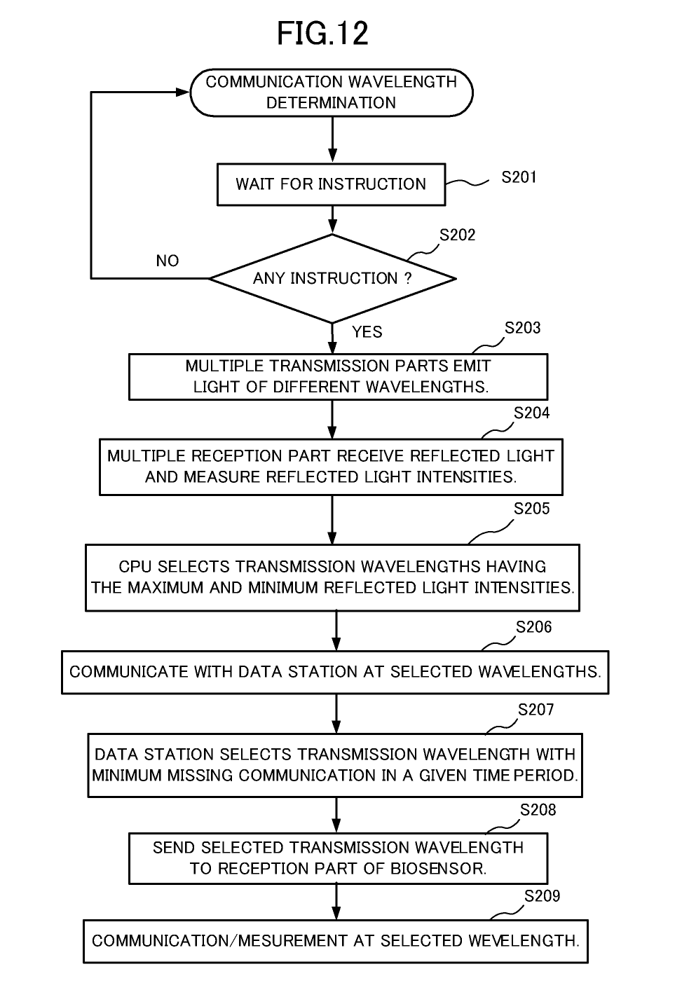

FIG. 12 is a flowchart of the biosensor system according to Modified Embodiment 3 of Embodiment 2. Specific schematic illustrations of the biosensor unit 1 and data station 2 according to Modified Embodiment 3 of Embodiment 2 will be similar to FIGS. 7 and 8. The Modified Embodiment 3 will be described in detail hereafter with reference to FIGS. 7, 8, 11, and 12.

A communication wavelength most suitable for the clothes C the user of the biosensor unit 1 is wearing is measured. For example, first, the user issues an instruction for measuring a communication wavelength through operation buttons 22 of the data station 2 (Step S201 and Step S202). This step can be repeated at given time intervals or each time the user changes the clothes C. Furthermore, the CPU 13 can be designed to perform this step automatically on a periodic basis even if there is no instruction from the user of the biosensor unit 1.

Then, multiple transmission parts (light emitting elements) 15 emit light of different wavelengths (Step S203). The multiple transmission parts (light emitting elements) 15 may emit light simultaneously or by turns in any order. Then, the light emitted from the light emitting elements is reflected by the clothes C as shown in FIG. 11. The intensity of the reflected light varies depending on the wavelength of light emitted by the multiple transmission parts (light emitting elements) 15. Furthermore, the intensity of the reflected light varies depending on the color and material of the clothes C the user is wearing because the intensity of absorbed light differs. Then, the respective corresponding reception parts 16 receive the reflected light and the intensities of the reflected light are measured (Step S204).

Among the intensities of the reflected light measured by the reception parts 16, for example, the transmission wavelengths having the maximum and minimum reflected light intensities are selected by the CPU 13 (Step S205). At the transmission wavelength having the maximum reflected light intensity, less light may be absorbed by the clothes C; however, light may not easily be transmitted through the clothes C, whereby it is presumably not desirable for communication with the data station 2. On the other hand, at the transmission wavelength having the minimum reflected light intensity, more light may be absorbed by the clothes C; however, light may easily be transmitted through the clothes C, whereby it is presumably desirable for communication with the data station 2. For this reason, wavelengths yielding two reflected light intensities are selected in this Modified Embodiment. The selection of wavelengths based on the reflected light intensity is not restricted to two wavelengths. For example, two wavelengths yielding two highest reflected light intensities and two wavelengths yielding two lowest reflected light intensities can be selected.

Communication with the data station 2 is performed at the wavelength selected as described above (Step S206). More specifically, in accordance with FIGS. 7 and 8 described above, communication from the transmission part 15 to the transmission/reception port 24 or station transmission/reception part 29 is performed. Then, at the data station 2, for example, the number of times of missing communication in a given time period (or missing communication time) is calculated and the transmission wavelength with minimum missing is selected (Step S207).

The selected transmission wavelength with minimum missing is the transmission wavelength most suitable for the biosensor system 101. Information on the selected transmission wavelength is sent from the data station 2 to the reception part 16 of the biosensor unit 1 (Step S208). Then, communication at the selected transmission wavelength is performed in use of the biosensor system 101 (Step S209). Using the biosensor system according to this Modified Embodiment, obstacles to optical communication such as the thickness and color of the clothes C can be reduced.

As described above, the biosensor unit and biosensor system according to Embodiment 2 provides stable, power-saving communication with less influence on the human body and no mutual influence with other electronic devices.

Furthermore, the biosensor unit and data station of the biosensor system according to Embodiment 2 can transmit/receive data to/from each other. In this way, it becomes possible to reset measurement conditions for measuring in the biosensor unit, calibrate the calibration curve, and/or set storing or retrieving of other data on an arbitrary basis, thereby realizing use of a biosensor system tailored to the user or high performance measurement.

Furthermore, having multiple transmission parts and reception parts, the biosensor unit is improved in directionality and performs stable data transmission/reception. Furthermore, with the signal amplifying part situated closer to the sensor part, influence of noise is reduced and highly accurate measurement is available.

Furthermore, with the base part of the biosensor unit being secured by the adhesive layer, the risk of the biosensor unit coming off can be reduced, whereby the user can use it continuously for a prolonged time period, which will reduce the burden on the user.

In the biosensor unit according to Embodiment 2, the reception part and adhesive layer are added to the biosensor unit according to Embodiment 1. However, either one of them may be added or the two of them may be added. The number, position, shape, and size of the additional reception part and adhesive layer are not restricted to the above embodiment and can be determined on an arbitrary basis. Whether the signal amplifying part is situated on the subcutaneously retained part is determined also on an arbitrary basis.

### Embodiment 3

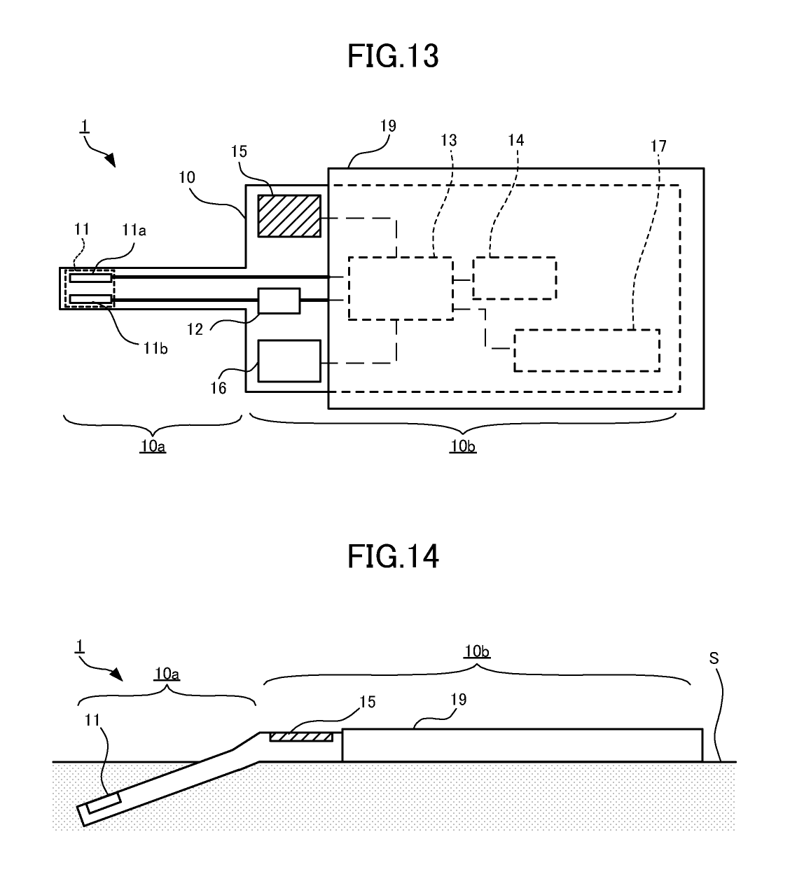

FIG. 13 is a schematic illustration showing the structure of the biosensor unit according to Embodiment 3 of the present invention. FIG. 14 is a schematic side view of the biosensor unit shown in FIG. 13. This biosensor unit has the same basic structure as the biosensor unit 1 according to Embodiment 1. However, a fixing member 19 is additionally provided.

The fixing member 19 is directly applied to the skin S to cover the base part 10b without any space between the skin S and biosensor unit 1. The fixing member 19 is one size larger than the base part 10b. Furthermore, it is applied to the skin S, leaving the transmission part 15 and reception part 16. With the fixing member 19 covering and fixing the base part 10b, the unevenness between the base part 10b and skin S is eliminated, whereby the risk of the base unit 10b being caught and the biosensor unit 1 being removed or coming off from the attached position will be reduced.

If the fixing member 19 covers the transmission part 15 and reception part 16, data transmission/reception may be interfered. Therefore, it is desirable to preliminarily situate the transmission part 15 and reception part 16 at the end of the base part 10b. The biosensor unit 1 will be caught most easily when there is a space between the base part 10b and skin S at the end of the biosensor unit 1 opposite to the sensor part 11. Therefore, it is desirable to cover that end with the fixing member 19 as much as possible. Therefore, it is preferable that the transmission part 15 and reception part 16 are provided on the base part 10b near the subcutaneously retained part 10a.

It is preferable that the fixing member 19 is mild to the skin S because it is directly applied to the skin S. For example, it can be formed by providing an adhesive layer such as hydrogel adhesive and silicon adhesive on an unwoven fabric base. More specifically, a medical tape, adhesive plaster, or Kinesio tape can be used. In such a case, the fixing member 19 similar in color to the skin S makes the worn biosensor unit 1 invisible.

When the fixing member 19 consists of a transparent film and has very little influence on optical data communication, the fixing member 19 can be applied to the skin S to cover the base part 10b. Therefore, the biosensor unit 1 can be designed without consideration for the positions of the transmission part 15 and reception part 16.

As described above, the biosensor unit according to Embodiment 3 provides stable, power-saving communication with less influence on the human body and no mutual influence with other electronic devices.

With the biosensor unit being secured to the skin by the fixing member, the risk of the biosensor unit coming off can be reduced, whereby the user can use it continuously for a prolonged time period, which will reduce the burden on the user. Particularly, the unevenness between the base part of the biosensor unit and the skin is eliminated, whereby the risk of the base part being caught and the biosensor unit being removed or coming off from the attached position will be reduced. Then, the user using the biosensor unit is not restricted in movement and experience less difficulty in movement.

In the biosensor unit according to Embodiment 3, a fixing member is additionally provided to the biosensor unit according to Embodiment 2. However, the fixing member can additionally be provided to the biosensor unit according to Embodiment 1 or to the biosensor units according the modified embodiments of Embodiment 2. It is possible to secure the biosensor unit to the skin by an adhesive layer and further cover it with a fixing member. The shape, size, position to attach, and material of the fixing member are not restricted to the above embodiment and can be determined on an arbitrary basis.

The biosensor unit and biosensor system according to this embodiment are given by way of example. The shape, size, functions of the functional parts, selection of materials, positions of the functional parts, number of transmission parts and reception parts of the biosensor unit can be determined on an arbitrary basis. The shape and function of the data station corresponding to the biosensor unit can also be determined on an arbitrary basis.

Furthermore, the biosensor system according to this embodiment can be provided with a given length of optical fiber continuously extended from the transmission part of the biosensor system and from the end of which data is wirelessly transmitted to the data station. Use of optical communication can decrease influence on the human body, diminish susceptibility to noise, and reduce influence on other electronic devices.

Furthermore, in the biosensor unit according to this embodiment, the battery part constitutes a primary battery. It can be rechargeable in a contact or noncontact manner.

Having described and illustrated the principles of this application by reference to one (or more) preferred embodiment(s), it should be apparent that the preferred embodiments may be modified in arrangement and detail without departing from the principles disclosed herein and that it is intended that the application be construed as including all such modifications and variations insofar as they come within the spirit and scope of the subject matter disclosed herein.

What is claimed is:

1. A biosensor unit, comprising:
a sensor part retained under the skin and continuously detecting signal values regarding a target substance in the specimen;
a calculation part converting said signal values to transmittable data; and
a transmission part wirelessly transmitting said data to an external device using infrared or visible light.

2. The biosensor unit according to claim 1, wherein the wireless transmission performed by said transmission part utilizes a light source that is an LED, organic EL, or semiconductor laser.

3. The biosensor unit according to claim 1, comprising a reception part receiving data from an external source.

4. The biosensor unit according to claim 3, comprising a plurality of said transmission parts and/or said reception parts.

5. The biosensor unit according to claim 4, wherein:
said plurality of transmission parts transmit said infrared or visible light having different wavelengths;
said reception parts receive light when said plurality of transmission parts each emit said infrared or visible light; and
said transmission parts transmit said data using a wavelength having the maximum light intensity and/or minimum light intensity received by said reception parts when the transmission parts transmit light.

6. The biosensor unit according to claim 1, comprising a signal amplifying part amplifying said signal values from said sensor part.

7. The biosensor unit according to claim 6, wherein said signal amplifying part is provided on said subcutaneously retained part that is retained under the skin.

8. The biosensor unit according to claim 1, constructed as an integrated item having said subcutaneously retained part that is retained under the skin and a base part that is placed on the skin surface.

9. The biosensor unit according to claim 8, wherein said base part comprises a fixing member that is applied to the skin so as to cover the base part.

10. The biosensor unit according to claim 8, wherein said base part comprises a sticky adhesive layer on the surface of the base part that faces the skin.

11. The biosensor unit according to claim 1, wherein a biocatalyst reactive with said target substance in the specimen is placed in said sensor part and said biocatalyst produces signals corresponding to the concentration of said target substance in the specimen through reaction with said target substance.

12. A biosensor system, comprising:
a biosensor unit comprising a sensor part retained under the skin and continuously detecting signal values regarding a target substance in the specimen, a calculation part converting said signal values to transmittable data, and a transmission part wirelessly transmitting said data to an external device using infrared or visible light; and
a data station comprising a reception part capable of receiving data transmitted from said biosensor unit.

13. The biosensor system according to claim 12, wherein said biosensor unit comprises a reception part receiving data from an external source.

14. The biosensor system according to claim 13, wherein said biosensor unit comprises a plurality of said transmission parts and/or said reception parts.

15. The biosensor system according to claim 14, wherein in said biosensor unit,
said plurality of transmission parts transmit said infrared or visible light having different wavelengths;
said reception parts receive light when said plurality of transmission parts each emit said infrared or visible light; and
said transmission parts transmit said data using a wavelength having the maximum light intensity and/or minimum light intensity received by said reception parts when the transmission parts transmit light.

16. The biosensor system according to claim 12, wherein said biosensor unit comprises a signal amplifying part amplifying said signal values from said sensor part.

17. The biosensor system according to claim 12, wherein said biosensor unit is constructed as an integrated item having said subcutaneously retained part that is retained under the skin and a base part that is placed on the skin surface.

18. The biosensor system according to claim 12, wherein a biocatalyst reactive with said target substance in the specimen is placed in said sensor part and said biocatalyst produces signals corresponding to the concentration of said target substance in the specimen through reaction with said target substance.
